(19) United States
(12) Patent Application Publication
(10) Pub. No.: US 2007/0232866 A1
(43) Pub. Date: Oct. 4, 2007

(54) Medical Patient Monitoring and Data Input Systems, Methods and User Interfaces

(75) Inventors: Paul James Nephin, Carleton Place (CA); Don Edward Waterman, Appleton (CA); John Herbert Schneider, Kanata (CA); Evan Bruce Trickey, Carleton Place (CA)

Correspondence Address: WOOD, PHILLIPS, KATZ, CLARK & MORTIMER, 500 W. MADISON STREET, SUITE 3800, CHICAGO, IL, 60661, US

(73) Assignee: Neptec Design Group Ltd.

(21) Appl. No.: 11/547,429

(22) PCT Filed: Mar. 31, 2005
(86) PCT No.: PCT/CA05/00476

§ 371 Date: Sep. 29, 2006

Related U.S. Application Data
(60) Provisional application No. 60/557,714, filed on Mar. 31, 2004.
(60) Provisional application No. 60/564,985, filed on Apr. 26, 2004.

(51) Int. Cl. A61B 5/00

(52) U.S. Cl. 600/300

(57) ABSTRACT

Systems, methods, and associated user interfaces for medical patient health monitoring systems are provided. Data entry at a patient system deployed at a patient location is facilitated through a soft keypad graphical element. The soft keypad graphical element defines user input areas of the display which correspond to respective alphanumeric inputs, and user inputs within any of the user input areas are detected. Detected user inputs may be displayed, stored in a memory, transmitted to a remote location, or otherwise processed. Patient-site monitoring system configuration mechanisms are also provided to support configuration of a patient system to operate with any of various peripheral devices. The presentation of prompts and other information to a patient is preferably controlled according to how the patient system has been configured, illustratively the types of peripheral devices in conjunction with which the patient system has been configured to operate.

### FIELD OF THE INVENTION

This invention relates generally to medical patient monitoring and, in particular, to interface and data input systems and methods for patient monitoring.

### BACKGROUND

Monitoring of medical patients after release from hospital or for ongoing assessment of a medical condition, for example, presents many challenges. Attending medical appointments at a health care facility may not be convenient for a patient, such as when a medical condition or injury affects a patient's mobility or ability to travel. Where a desired or required level of monitoring involves relatively frequent determination of vital signs or other indicators of patient health, such visits to a health care facility may not be feasible.

In the field of remote health care monitoring, several systems are currently available. In one such system, predetermined health care questions and medication reminders are stored on an electronic device which is deployed at a patient site, typically the patient's home. The patient is prompted to answer the questions, and possibly to take medications or perform other tasks such as taking readings using any of a number of medical devices, including a stethoscope or glucometer, for example. Answers to the questions and readings from the devices may then be transmitted to a remote location for subsequent retrieval and analysis by a health care provider.

Although this type of remote monitoring system provides an alternative to attendance of medical appointments for patient monitoring, currently available systems have significant restrictions.

For example, current patient site electronic devices do not provide for convenient manual data input where a patient is required to input a reading from a medical device, for instance. In one known patient device, physical directional keys are provided on the patient device for input of such readings. The patient must then use the directional keys to adjust a value to reflect a device reading. The repeated operation of the directional keys may be difficult for older patients or those having certain medical conditions.

Current patient site electronic devices are also typically pre-configured, and not dynamically configurable for operation with any of multiple peripheral devices. Custom monitoring systems, including predetermined peripheral devices, are normally deployed at a patient site. Adaptation of such systems for changing patient needs thus tends to be difficult.

### SUMMARY OF THE INVENTION

Embodiments of the invention address at least some of the above disadvantages of current remote patient monitoring systems, by providing improved user input mechanisms. User input interfaces according to embodiments of the invention are more intuitive and provide for more efficient manual data input than known solutions.

Further embodiments of the invention relate to dynamically configurable patient health monitoring devices, which are more easily adaptable for operation in conjunction with any of multiple peripherals.

According to one aspect of the invention, there is provided a system for monitoring health conditions of a medical patient. The system includes a display and a user input manager. The user input manager is operatively coupled to the display and configured to display on the display a user prompt for medical information and a keypad graphical element defining user input areas of the display, each of the user input areas corresponding to a respective alphanumeric input, and to detect an input of the medical information by a user within any of the user input areas.

The user input manager may be implemented, for example, using a processor.

In some embodiments, the user input manager is configured to detect multiple user inputs in response to the user prompt. The user inputs may represent portions of the medical information, in which case the user input manager is preferably further configured to detect that user input is complete. The user inputs may then be parsed into the portions of the medical information, based on delimiter characters separating the portions of the medical information for instance.

Detected user inputs may be displayed, stored in a memory, transmitted to a remote location, or otherwise processed.

A method of monitoring health conditions of a medical patient is also provided, and includes displaying on a display a user prompt for medical information and a keypad graphical element defining user input areas of the display, each of the user input areas corresponding to a respective alphanumeric input, and detecting an input of the medical information by a user within any of the plurality of user input areas.

Methods according to other embodiments of the invention may include additional operations, such as any of those described briefly above in the context of a patient monitoring system.

The present invention also provides, in another broad aspect, a graphical user interface for an electronic device which is for monitoring health conditions of a medical patient. The graphical user interface includes a first graphical element comprising a user prompt for medical information, and a second graphical element defining user input areas of the display, each of the user input areas corresponding to a respective alphanumeric input.

In some embodiments, the graphical user interface also includes a graphical element defining a further user input area for indicating a completion of user input in response to the user prompt, a graphical element indicating a current user input, or both.

A system in accordance with another aspect of the invention includes a display, a memory, and a user input manager. The memory is for storing user prompts for user information and respective ranges of responses for any of the user prompts having an associated range of responses, and the user input manager is configured to retrieve from the memory and display on the display a user prompt, to determine whether the retrieved user prompt has an associated range of responses, and to display on the display, based on the determination, a keypad graphical element defining alphanumeric user input areas of the display or a range input graphical element defining a user input area of the display and a user input range comprising the associated range of responses.

A related method is also provided, and includes operations of displaying a user prompt on a display, determining a type of input solicited from a user by the user prompt, and displaying on the display, based on a result of the determining, a graphical element defining a user input area of the display. The displayed graphical element is selected from a keypad graphical element defining a plurality of alphanumeric user input areas of the display and a range input graphical element defining a user input area of the display and a user input range comprising a range of responses to the user prompt.

A further aspect of the invention provides a system for monitoring health conditions of a medical patient. The system includes a display, an interface, and a controller. The controller, which may be implemented using a processor for instance, is operatively coupled to the display and to the interface for displaying health care instructions on the display, and for dynamically configuring the interface for operation with a peripheral device.

In one embodiment, the health care instructions comprise health care instructions associated with the peripheral device. The controller preferably determines whether the interface has been configured for operation with the peripheral device, and displays on the display the health care instructions associated with the peripheral device where the interface has been configured for operation with the peripheral device. This determination by the controller may be made, for example, by detecting connection of the peripheral device to the interface.

The interface may include a single configurable interface or multiple interfaces. According to one embodiment, the or each interface is configurable for operation with any of a number of peripheral devices, which are preferably medical devices such as a blood pressure meter, an oximeter, a glucometer, a weigh scale, and a stethoscope.

Medical readings or other information collected in accordance with displayed health care instructions may be transmitted to a remote system for storage and/or processing. Responsive to an instruction received from a remote system, for example, the controller may transmit readings or information to that particular remote system.

Another aspect of the invention provides a method of monitoring health conditions of a medical patient. The method includes operations of determining whether a peripheral device has been enabled at a patient monitoring system, and displaying at the patient monitoring system health care instructions comprising at least one of: health care instructions associated with the peripheral device and health care instructions other than the health care instructions associated with the peripheral device, based on the determination.

A related system for monitoring health conditions of a medical patient includes means for determining whether a peripheral device has been enabled at a patient monitoring system, and means for displaying at the patient monitoring system health care instructions comprising at least one of: health care instructions associated with the peripheral device and health care instructions other than the health care instructions associated with the peripheral device, based on the determination.

Other aspects and features of embodiments of the present invention will become apparent to those ordinarily skilled in the art upon review of the following description of the specific embodiments of the invention.

### BRIEF DESCRIPTION OF THE DRAWINGS

Examples of embodiments of the invention will now be described in greater detail with reference to the accompanying diagrams, in which:

FIG. 1 is a block diagram of a patient monitoring system in which embodiments of the invention may be implemented;

FIG. 2 is a block diagram of an illustrative example patient system;

FIG. 3 is a representation of a GUI for a patient system;

FIG. 4 is a representation of another GUI for a patient system;

FIG. 5 is a flow diagram of a data input method according to an embodiment of the invention; and

FIG. 6 is a flow diagram of a method of monitoring health conditions of a medical patient according to another embodiment of the invention.

### DETAILED DESCRIPTION OF PREFERRED EMBODIMENTS

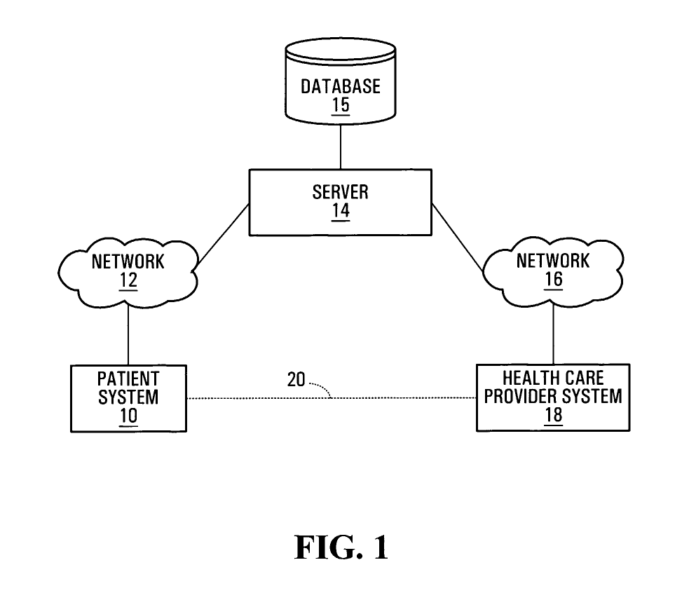

FIG. 1 is a block diagram of a patient monitoring system in which embodiments of the invention may be implemented. The system of FIG. 1 includes a patient system 10, a communication network 12, a server 14, a database 15, a communication network 16, a health care provider system 18, and a communication link 20. It should be appreciated, however, that the particular system shown in FIG. 1 is intended for illustrative purposes only, and that the invention is in no way limited thereto.

For example, it will become apparent from the following description that embodiments of the invention are not dependent upon any particular communication schemes, protocols, or network topologies. Those skilled in the art will appreciate that virtually any communication technique may be used to provide for communication between the system components shown in FIG. 1. It should also be appreciated that embodiments of the invention may be implemented in systems with further or fewer components than those explicitly shown in FIG. 1. A patient monitoring system may include many patient systems 10, multiple health care provider systems 18, and even multiple servers 14 or databases 15.

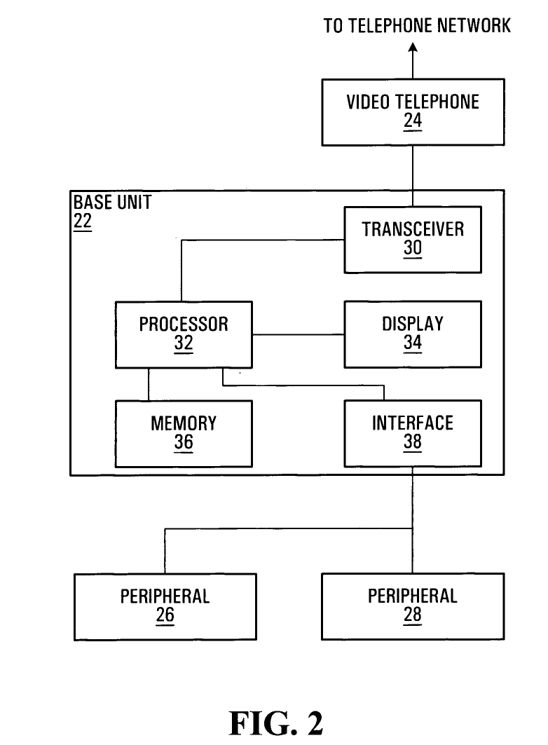

The patient system 10 is an electronic device intended for deployment at a patient site such as in the home of a patient. An example of such a system in described in further detail below in conjunction with FIG. 2.

The network 12 is a communication network through which the patient system 10 communicates with the server 14. In one embodiment, the network 12 is a public telephone network, although other types of communication networks and links will be apparent to those skilled in the art. It is also contemplated that different patient systems 10 may communicate with the server 14 through different networks or different types of networks. Given the sensitivity of medical information, a secure transfer mechanism is preferably implemented between the patient system 12 and the server 14.

The server 14 is a remotely accessible computer system with which the patient system 10 and the health care provider system 18 may establish communications and exchange information, possibly in both directions between the server 14 and each of the patient station 10 and the health care provider system 18. Information stored in the database 15 at the server 14 may thereby be made accessible to the patient system 10 and the health care provider system 18, and information transmitted to the server 14 from the patient system 10 or the health care provider system 18 is preferably stored in the database 15.

The network 16 may be the same type, or even the same network, as the network 12, or a different type of network. In one embodiment, the network 12 is a telephone network, and the network 16 is a data communication network such as the Internet. Where the server 14 and the health care provider system 18 are co-located, at a hospital for instance, the network 16 may be a local area network (LAN). Different health care provider systems 18 may communicate with the server 14 through different networks or types of networks, and communications between the health care provider system 18 and the server 14 are preferably secure, using a Virtual Private Network (VPN) connection, for example.

The health care provider system 18 is a computer system, illustratively a personal computer, through which a health care provider interacts with the server 14 and the patient system 10 so as to remotely monitor one or more medical patients in their care.

Although shown as a direct connection, the communication link 20 may also be a network connection, through a telephone network, for example. The link 20 enables interaction between a health care provider and a patient, to conduct a remote, substantially real-time, medical assessment or “televisit” session. The health care provider is thereby able to actively assess current medical conditions of the patient without physically visiting the patient or requiring the patient to travel to a health care facility. For example, videotelephones or some other video conferencing equipment may be implemented at both the patient system 10 and the health care provider system 18 so that a televisit may include visual assessment of medical conditions.

According to one possible operating scheme, an account is created for a health care provider on the server 14. The health care provider, using the provider account, then configures patient accounts or profiles, including patient identification information, medical conditions, medication reminders, alert conditions for which a medical alert will be generated for the patient, the health care provider, or another health care provider for example, and a set of health questions to be used to periodically prompt the user for medical information. Access to patient profile creation and management functions may be provided through the health care provider system 18, and/or through other systems such as a local workstation or administrator terminal which is operatively coupled to the server 14.

Any or all patient information in a patient profile is preferably then loaded onto the patient station 10. For an initial deployment of the patient station 10, loading may be performed through a physical local connection to the server 14, whereas remote updates through the network 12 may be preferred where the patient system has already been deployed at the patient location. At least any medical reminders and questions are preferably loaded onto the patient system 10. Other patient profile information may also be loaded, at the discretion of the health care provider, for example.

Considering the patient system 10 in more detail, after the patient system 10 has been configured with reminders and/or questions, the patient system 10 presents the reminders or questions to the patient. FIG. 2 is a block diagram of an illustrative example of the patient system 10.

The patient system of FIG. 2 includes a base unit 22, which effectively provides an operating platform for the patient system and may operate with or without an optional videotelephone 24 and optional peripherals 26, 28. The base unit 22 includes a transceiver 30, a processor 32, a display 34, a memory 36, and an interface 38. However, the present invention is not restricted to the particular implementation of a patient system shown in FIG. 2. Embodiments of the invention disclosed herein may be applied to patient systems which include fewer, further, or different components than those specifically shown in FIG. 2, with different interconnections therebetween.

The transceiver 30 enables information to be transmitted from and received by the base unit 22, although as described above, only a transmitter may be provided where information need only be sent from a patient system to a remote server such as the server 14 for instance. Those skilled in the art will appreciate that many different types of transceiver are suitable for use as the transceiver 30 in the base unit 22, including those for wired or wireless communications.

Although the videotelephone 24 is an optional component, the transceiver 30 is preferably compatible with the videotelephone 24. Such compatibility allows for deployment of substantially the same base unit 22, which may be configured, at deployment or subsequently, for operation with or without the videotelephone 24. In this manner, a videotelephone may be added to a patient system when required or removed from the patient system when visual monitoring of the patient is no longer required. Alternatively, different types of transceivers may be provided for respective connection to the videotelephone 24 and some other device through which communications may be established between the patient system and a remote system such as the server 14 or the health care provider system 18 (FIG. 1).

The processor 32 may be, for example, a microprocessor which is configured to execute patient system software for performing the operations described in further detail below. Normally, patient system software will be stored in the memory 36 and executed by the processor 32. Other implementations of the processor 32 are also contemplated. Display controllers, Application Specific Integrated Circuits (ASICs), and microcontrollers are illustrative examples of other types of component using which the functions of the processor 32, or at least the user input functions disclosed herein, may be provided. It should thus be apparent that embodiments of the invention may be implemented using software for execution by a processor, hardware, or some combination thereof.

As will be apparent, the display 34 is a component that displays information to a patient. A liquid crystal display (LCD) is one common type of display for an electronic device such as the patient system. In a preferred embodiment, the display is a touchscreen which senses physical contact. According to another embodiment, the display 34, or possibly a separate component, detects an input stylus, such as a patient's finger or a component supplied with or configured for operation with the patient system, in proximity to an input area of the display 34.

The memory 36 is preferably a solid state memory. Other types of memory, such as a hard disk drive or a memory device which operates in conjunction with a removable recording medium, for example, may also be used as the memory 36. In another embodiment, the memory 36 includes more than one type of memory. As will become apparent from the following description of the operation of the patient system, the memory 36 may store any reminders and questions which have been configured for the patient, patient profile information, and inputs received from the patient. The memory 36 also preferably stores software to be executed by the processor 32, which may include operating system software and application software. Patient monitoring may instead be integrated within operating system software, for example.

The interface 38, although shown as a single component, may include multiple interfaces, and even different types of interface compatible with corresponding interfaces (not shown) in the peripherals 26, 28. Examples of the interface 38 include Bluetooth™ modules and other wireless communication interfaces, infrared ports, and Universal Serial Bus (USB) ports and other types of serial or parallel data ports, although the invention is in no way restricted to these types of interfaces. The interface 38 may also provide for further functions than communications with the peripherals 26, 28, such as power connections for providing power to operate the peripherals 26, 28 or to recharge batteries in the peripherals 26, 28. As described briefly above, the peripheral devices 26, 28 are optional. However, a base unit 22 which incorporates the interface 38 may be used with or without the peripherals 26, 28, to provide a dynamically configurable base unit 22.

The peripherals 26, 28 are preferably medical devices which may be used to collect health information or vital signs from the patient, including a blood pressure meter, an oximeter, a glucometer, a weigh scale, or a stethoscope, for instance. Other types of medical devices will be apparent to those skilled in the art.

Additional or different components, not shown in FIG. 2, may also be provided in patient equipment. For instance, a speaker or other suitable audio output device would allow audio presentation of user prompts, instructions, and other information at a patient system. A translator may also be provided at a patient system to translate text-based user prompts into corresponding audio prompts. A software module or utility which translates a user prompt data format, illustratively ASCII, into an audio signal format represents one example implementation of a translator.

Other possible variations of the patient system of FIG. 2 will be apparent to those skilled in the art.

As described briefly above, a patient system preferably presents a patient with health questions configured by a health care provider and loaded into the memory 36 of the patient system. The health questions prompt a user for information. Embodiments of the invention facilitate the input of such information by the patient through GUIs displayed on the display 34.

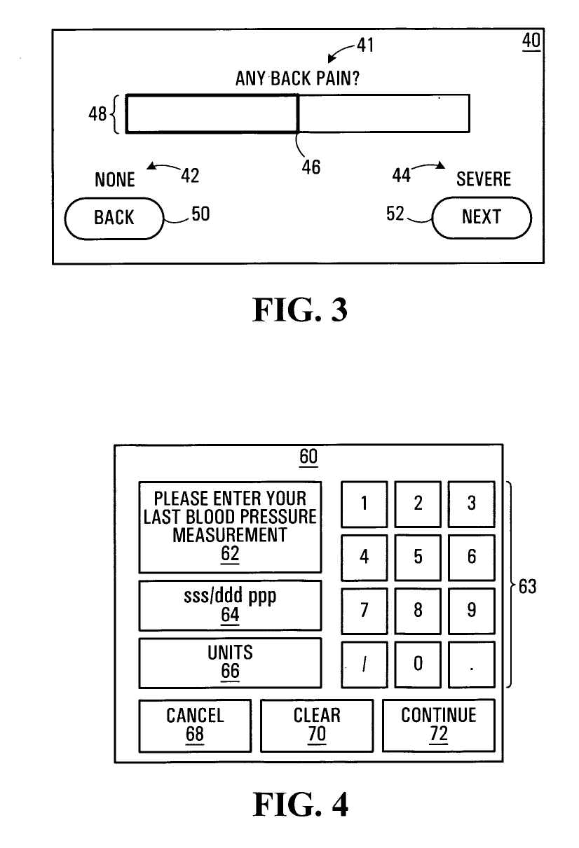

FIG. 3 is a representation of a GUI for a patient system. The GUI 40, a screen which is displayed on the display 34, includes a health question or prompt 41, a graphical element 48 which indicates a user input range with the indicators 42, 44 and a current user input at 46, and control graphical elements 50, 52 which allow a patient to complete or cancel a current input. The GUI of FIG. 3 is an illustrative example of a GUI that may be provided at a patient system, and is not intended to limit the invention. Other layouts, shapes, sizes, and text, for example, may be used for a patient system GUI.

Referring now to both FIGS. 2 and 3, the processor 32 is configured to display on the display 34 a graphical element 48 defining a user input area of the display 34 and a user input range, and also to detect an input within the user input area. The graphical element 48 is then modified, as shown at 46, to indicate the detected input. Initially, the graphical element 48 is preferably one color or pattern, and is modified when an input is detected to indicate the detected input, such as when the patient touches the display 34 within the user input area defined by the graphical element 48.

To change the current input, the patient may touch a different part of the user input area, and the graphical element 48 is again modified to indicate the new detected input. When the patient has completed the current input, a further input may be made using the “Next” element 52. Alternatively, another type of input, a physical key for example, may be provided to indicate the completion of user input. The detected input is then accepted as a user input and may be translated into a number or some other format for further processing by the patient system. The detected input, a translated version of the input, or both, may be displayed to the patient, stored in the memory 36, and transmitted from the patient system. For a non-numerical user input range as shown in FIG. 3, a detected input is preferably translated into a numerical value.

As shown at 41, a user prompt in the form of a health question is also displayed as a graphical element, which may be a separate graphical element. Those skilled in the art will appreciate that the range graphical element 48 and the prompt graphical element 41 may instead be the same graphical element. Similarly, the indicators 42, 44 and the control elements 50, 52 may also be provided as separate graphical elements or part of the same graphical element. Thus, the entire screen 40 may be one or more graphical elements. The particular form of the graphical element will depend upon the type of software used at the patient system, and is a matter of design. The invention is in no way limited to any specific number or type of graphical elements.

It will be apparent from FIG. 3 that the user input range represents a range of responses to the user prompt. The user prompt and its corresponding range of responses may be stored in the memory 36 and retrieved by the processor 32. More than one user prompt and corresponding ranges of responses may be stored in the memory 36. In this case, a next user prompt and range if applicable are displayed upon completion of user input in response to a current user prompt. As not every user prompt, such as a reminder, would necessarily require a user input or have an associated range of responses, completion of user input in response to a current prompt may result in the display of another response range. The current user prompt, or possibly a previous user prompt, and corresponding range if applicable may be displayed when a current input is cancelled. An input using the “Back” element 50 preferably results in display of a previous user prompt. Although not explicitly shown in FIG. 3, a “Cancel”, “Clear” or “Reset” element may be provided to clear a current input and re-display a current user prompt. Other control inputs, such as to skip or delay a response to a user prompt may also be provided.

Detection of user inputs may be enabled, for instance, by using a touchscreen as the display 34 such that physical contact of a user input area is detected to thereby detect an input. User inputs may instead be detected by sensing proximity of a stylus to the user input area. Other suitable input detection mechanisms, including those in which input detection is performed by a component or element that is separate from the display 34, will also be apparent to those skilled in the art.

FIG. 4 is a representation of another GUI for a patient system. The GUI 60, like the GUI 40, is a screen which is displayed on the display 34. A health question or prompt is presented in the GUI 60 at 62, and a keypad graphical element 63 defines a plurality of user input areas corresponding to respective alphanumeric inputs. A current user input, which in this example also provides an indication of input format, is displayed at 64, units of the input are displayed and possibly selectable using the graphical element 66, and control graphical elements 68, 70, 72 allow a patient to cancel, clear, or complete a current input. The various displayed graphical elements may be provided as one or more graphical components or models. The GUI of FIG. 4, like the GUI of FIG. 3, is intended solely for illustrative purposes.

Whereas the GUI of FIG. 3 provides for convenient and intuitive user input for user prompts with associated ranges of possible responses, the GUI of FIG. 4 facilitates input of alphanumeric information, a blood pressure reading with systolic and diastolic pressures (sss/ddd) and a pulse rate (ppp) in the particular illustrative example of FIG. 4.

With reference now to both FIGS. 2 and 4, the processor 32 displays the user prompt for medical information at 62 and the keypad graphical element 63 and detects an input of the medical information within any of the user input areas defined by the keypad graphical element 63. The detected input is then displayed at 64. Any of the detection mechanisms described above may be employed in conjunction with the GUI 60.

Where more than one digit of input may be necessary, multiple inputs may be detected in response to the user prompt. For example, detected inputs may represent portions of the medical information, such as the systolic pressure, diastolic pressure, and pulse rate in FIG. 4. The processor 32 then preferably detects that inputs in response to a current user prompt have been completed, by detection of an input using the “Continue” element 72. The complete input may then be parsed into the portions of the medical information. Delimiter characters, such as the slash character in FIG. 4, may be used to separate the portions of the medical information.

Further processing of detected inputs and subsequent display of other stored user prompts and possibly range or keypad graphical elements upon completion of user input may be performed substantially as described above.

Although the keypad graphical element 63 includes only numbers and two operators, further characters, including alphabetic characters and other operators may also or instead be provided.

A soft keypad such as shown in FIG. 4 provides several advantages over current user input mechanisms for health monitoring devices. For example, it allows the patient to enter inputs in a one-step operation instead of adjusting a current input using physical directional keys. Such a keypad also allows a patient to enter complex data strings that are subsequently parsed and processed by the processor 32 and the software it executes.

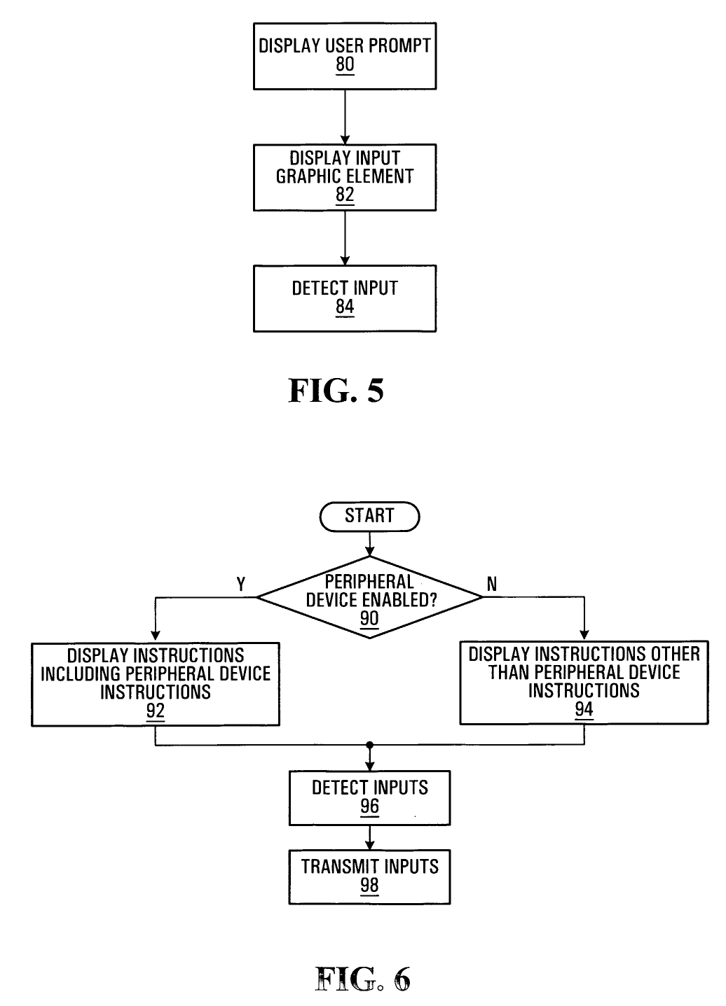

FIG. 5 is a flow diagram of a data input method. The method includes displaying a user prompt at 80, displaying an input graphical element at 82, and detecting a user input which is made using the graphical element at 84. The display operations at 80 and 82 may be performed at substantially the same time or separately. The input graphical element displayed at 82 may be a range graphical element such as in FIG. 3 or a keypad graphical element such as in FIG. 4. In a further embodiment, the patient system processor 32 (FIG. 2) may determine the type of input required in response to a user prompt and then select and display the graphical element associated with that type of input. For example, a field, flag, or data structure may be stored in the memory 36 with each user prompt to indicate whether any input is required in response to the user prompt, and if so, whether that input has an associated range. A range graphical element is then displayed for user prompts requiring an input within a specified range, and a keypad is displayed for required non-range inputs.

As will be apparent from the foregoing, the flow diagram of FIG. 5 represents a general method that relates to both of the GUIs shown in FIGS. 3 and 4. A complete patient monitoring method may include further operations, such as translation, storage, parsing, and transmission of inputs as described above. Other embodiments of the invention may also involve fewer or different operations than those shown in FIG. 5, and/or operations which are performed in a different order than explicitly shown.

Various user input mechanisms in accordance with embodiments of the invention have been described above. Further embodiments of the invention relate to configuration of a patient system.

With reference again to FIG. 2, configuration of a patient system for operation with peripheral devices may involve loading a new set of instructions to the base unit 22, illustratively as software in the memory 36, to support operation in conjunction with peripheral devices 26, 28. New instructions may include prompts or reminders for a patient to take medical readings using the peripheral devices. Collection of readings from the peripheral devices and transmission of readings to a remote system such as the server 14 or the health care provider system 18 are preferably also enabled in the base unit 22 during its configuration for peripheral devices, by loading software into the memory 36 of the base unit 22, for example.

Although configuration of peripheral devices may be accomplished through new instruction and software loads, it should also be appreciated that a patient system may include appropriate instructions and software for all supported peripheral devices. In this case, functions associated with a particular peripheral device are activated or invoked only when that peripheral device is installed at the patient system.

In respect of dynamic configuration of peripherals, the processor 32 may be considered one possible implementation of a controller, which displays health care instructions on the display 34 and dynamically configures the interface 38 for operation with peripheral devices 26, 28.

The health care instructions may include health care instructions associated with a particular peripheral device, the peripheral device 26 for instance. In this case, the controller preferably determines whether the interface 38 has been configured for operation with the peripheral device 26, and if so, displays on the display 34 the health care instructions associated with the peripheral device 26. This determination may be made, for example, by detecting whether the peripheral device 26 has been connected or coupled to the interface 38. More generally, the type of health care instructions displayed to a patient on the display 34 may be based on the determination as to how the interface 38 has been configured, and/or the types of peripheral device 26, 28 to which it has been connected.

Medical readings or other information collected in accordance with displayed health care instructions may be transmitted to a remote system, such as the server 14 and/or the health care provider system 18 (FIG. 1) for storage, processing, or both. In some embodiments, transmission of readings or information is responsive to an instruction received from a remote system. During a televisit session, for example, a patient system may receive instructions and possibly medical device control signals from a health care provider system. Subsequent medical readings are then preferably transmitted to the health care provider system.

FIG. 6 is a flow diagram of a method of monitoring health conditions of a medical patient according to an embodiment of the invention. At 90, a determination is made as to whether a peripheral device has been enabled at a patient monitoring system, illustratively by configuring an interface. Health care instructions including instructions associated with the peripheral device are displayed at 92 where the peripheral device has been enabled at a patient monitoring system. Otherwise, health care instructions other than the health care instructions associated with the peripheral device are displayed at 94. It should be noted that the display of health care instructions associated with a peripheral device at 92 is not intended to exclude other health care instructions. Both types of health care instruction may be displayed when a peripheral device has been enabled.

As described above, a patient system may also perform additional operations. Input of information by the patient, or from the peripheral device if enabled, are detected at 96, and detected inputs are transmitted to a remote system at 98. The remote system may be a server, a health care provider system, or some other system. Inputs detected at 98 may also be stored at a patient system. Although a transmission operation at 98 is shown as immediately following detection at 96, inputs may be stored for later transmission at 98.

The method shown in FIG. 6 is presented solely for illustrative purposes; the invention is in no way limited thereto. Methods according to other embodiments of the invention may include further, fewer, or different operations performed in a different order. For example, the determination at 90 may be made after instructions which are not associated with the peripheral device have been displayed.

What has been described is merely illustrative of the application of the principles of the invention. Other arrangements and methods can be implemented by those skilled in the art without departing from the spirit and scope of the present invention.

For example, although systems are described above primarily in the context of a processor which executes software in which techniques according to embodiments of the invention have been implemented, other embodiments may instead be implemented with more than a single processor or physical component. The operations disclosed herein may be performed, for example, in separate components or devices, or in other types of components than a processor. Thus, references herein to a processor performing various user input-related functions and configuration functions should be interpreted accordingly. In effect, the processor 32 in FIG. 3 represents one possible implementation of a user input manager, and as described above, one possible implementation of a controller.

Similarly, it should be appreciated that components are shown within particular blocks or systems solely for illustrative purposes, and that the functionality disclosed herein may be supported with other system configurations, with different division of functions between system components.

In addition, embodiments of the invention have been described above primarily in the context of systems, methods, and GUIs. Other implementations are also possible, as instructions stored on a machine-readable medium, for instance.

1. A system for monitoring health conditions of a medical patient, comprising:
a display; and
a user input manager operatively coupled to the display and configured to display on the display a user prompt for medical information and a keypad graphical element defining a plurality of user input areas of the display, each of the plurality of user input areas corresponding to a respective alphanumeric input, and to detect an input of the medical information by a user within any of the plurality of user input areas.

2. The system of claim 1, wherein the user input manager is implemented using a processor.

3. The system of claim 1, wherein the user input manager is configured to detect a plurality of inputs by the user in response to the user prompt.

4. The system of claim 3, wherein the plurality of inputs comprise portions of the medical information, and wherein the user input manager is further configured to detect a further input by the user indicating that user input is complete, and to parse the plurality of inputs into the portions of the medical information in response to detection of the further input.

5-6. (canceled)

7. The system of claim 1, further comprising:
a memory,
wherein the user input manager is further configured to store the detected input in the memory.

8. The system of claim 7, wherein the user prompt comprises a user prompt stored in the memory, and wherein the user input manager is further configured to retrieve the user prompt from the memory.

9. (canceled)

10. The system of claim 1, further comprising a transmitter configured to transmit the detected input.

11. The system of claim 1, wherein the plurality of user input areas comprises user input areas corresponding to at least one of: numeric inputs and alphabetic characters.

12. The system of claim 1, wherein the display comprises a touchscreen, and wherein the user input manager is configured to detect physical contact of the user input area of the touchscreen to thereby detect the input.

13. The system of claim 1, wherein the user input manager is configured to detect proximity of a stylus to the user input area to thereby detect the input.

14. The system of claim 1, wherein the medical information comprises a reading from a medical device.

15. A method of monitoring health conditions of a medical patient, comprising:
displaying on a display a user prompt for medical information and a keypad graphical element defining a plurality of user input areas of the display, each of the plurality of user input areas corresponding to a respective alphanumeric input; and
detecting an input of the medical information by a user within any of the plurality of user input areas.

16. The method of claim 15, further comprising:
detecting completion of a plurality of inputs by the user in response to the user prompt; and
processing the plurality of inputs in response to detecting the completion.

17-18. (canceled)

19. The method of claim 15, wherein the user prompt comprises one of a plurality of user prompts, further comprising:
detecting a cancel input by the user; and
displaying on the display another one of the plurality of user prompts in response to detecting the cancel input.

20-21. (canceled)

22. A graphical user interface for an electronic device, the electronic device for monitoring health conditions of a medical patient, the graphical user interface comprising:
a first graphical element comprising a user prompt for medical information; and
a second graphical element defining a plurality of user input areas of the display, each of the plurality of user input areas corresponding to a respective alphanumeric input.

23. The graphical user interface of claim 22, further comprising:
a third graphical element defining a further user input area for indicating a completion of user input in response to the user prompt.

24. The graphical user interface of claim 22, further comprising:
a third graphical element indicating a current user input.

25. A system comprising:
a display;
a memory for storing user prompts for user information and respective ranges of responses for any of the user prompts having an associated range of responses; and
a user input manager configured to retrieve from the memory and display on the display a user prompt, to determine whether the retrieved user prompt has an associated range of responses, and to display on the display, based on the determination, a keypad graphical element defining a plurality of alphanumeric user input areas of the display or a range input graphical element defining a user input area of the display and a user input range comprising the associated range of responses.

26. The system of claim 25, wherein the user input manager is further configured to detect an input by a user within the user input area of the range input graphical element, and to modify the range input graphical element to indicate the detected input.

27. The system of claim 25, wherein the user input manager is further configured to detect an input of the medical information by a user within any of the user input areas of the keypad graphical element or the range input graphical element, and to display on the display the detected input.

28. A method comprising:
displaying a user prompt on a display;
determining a type of input solicited from a user by the user prompt; and
displaying on the display, based on a result of the determining, a graphical element defining a user input area of the display, the graphical element being selected from a keypad graphical element defining a plurality of alphanumeric user input areas of the display and a range input graphical element defining a user input area of the display and a user input range comprising a range of responses to the user prompt.

29-44. (canceled)
